(19) United States
(12) Patent Application Publication
(10) Pub. No.: US 2007/0196498 A1
(43) Pub. Date: Aug. 23, 2007

(54) Megestrol acetate suspension

(76) Inventor: Bernard Charles Sherman, Toronto (CA)

Correspondence Address: NIXON & VANDERHYE, PC, 901 NORTH GLEBE ROAD, 11TH FLOOR, ARLINGTON, VA, 22203, US

(21) Appl. No.: 11/357,996

(22) Filed: Feb. 22, 2006

(51) Int. Cl. A61K 9/14; A61K 31/56

(52) U.S. Cl. 424/489; 514/169

(57) ABSTRACT

Suspension comprising spray-dried particles which comprise megestrol acetate and a surfactant.

### BACKGROUND

Megestrol acetate is a synthetic progestin. It is sold in the United States and elsewhere under the tradename Megace™ in a suspension of strength 40 mg/mL.

Recently an improved suspension has been introduced into the U.S. market under the tradename Megace ES™. Megace ES™ has a strength of 125 mg/mL. It is said to have higher bioavailability than Megace™ after oral administration.

Megace ES™ suspension is made in accordance with the teaching of U.S. patent application Ser. No. 10/412,669. This document discloses that the higher bioavailability is achieved by using megestrol acetate in nanoparticles, which are defined as particles with an effective average size of less than about 2000 microns.

Micronizing to nanoparticle size is an expensive process. It is thus the objective of the present invention to enable suspensions that have improved bioavailability without requiring the use of the megestrol acetate in the form of nanoparticles.

### DESCRIPTION OF THE INVENTION

It has been found that the rate of dissolution of megestrol acetate in water, and thus the bioavailability of a megestrol acetate suspension, can be improved substantially by using megestrol acetate in the form of spray-dried particles comprising megestrol acetate and a surfactant.

The spray-dried patents are made by dissolving megestrol acetate and a surfactant in volatile solvent and spray-drying.

A preferred surfactant is sodium lauryl sulfate.

™ Registered Trademark

The amount of surfactant is preferably from about 5 percent to about 50 percent by weight, is more preferably from about 10 percent to about 30 percent by weight, and is most preferably about 20 percent by weight.

The spray-dried particles are incorporated into a suspension by blending the particles into an aqueous medium comprising, in addition to water, other usual suspension excipients such as sweeteners, flavours, preservatives, buffers, thickeners and surfactants.

The invention will be better understood from the following examples which are intended to be illustrative and not limiting.

### Example 1

Megestrol acetate and sodium lauryl sulfate were dissolved in a mixture of methanol and methyl chloride in proportions as follows:

| | |
|---|---|
| Megestrol acetate | 100 parts |
| Sodium Lauryl Sulfate | 25 parts |
| Methocel | 625 parts |
| Methyl Chloride | 250 parts |

The solution was spray dried.

### Example 2

A suspension was made by blending together at high shear ingredients as follows:

| | Quantity per L | |
|---|---|---|
| Megestrol Acetate Spray Dried | 156.25 | g |
| (from example 1 ) | | |
| Docusate Sodium | 1.35 | g |
| Citric Acid Phosphate | 1.30 | g |
| Xanthan Gum | 2.00 | g |
| Sodium Citrate Dihydrate | 0.50 | g |
| Sodium Benzoate | 1.60 | g |
| Sucrose | 43.0 | g |
| Natural Lemon-Lime Flavour | 1.60 | g |
| Water | qs to 1 | L |

156.25 g of spray dried particles from example 1 comprise 125 g of megestrol acetate. The strength of the suspension is thus 125 mg/mL.

It was found that, on dispersion in water, the dissolution rate of the megestrol acetate in this suspension is higher than that of Megace™ and comparable to that of Megace ES™.

1. A pharmaceutical suspension comprising spray-dried particles which comprise megestrol and a surfactant.

2. A suspension of claim 1 wherein the surfactant is sodium lauryl sulfate.

3. A suspension of claim 1 wherein the amount of surfactant in the particles is from about 5 percent to about 50 percent by weight.

4. A suspension of claim 1 wherein the amount of surfactant in the particles is from about 10 percent to about 30 percent by weight.

5. A suspension of claim 1 wherein the amount of surfactant in the particles is about 20 percent by weight.
